(19) United States
(12) Patent Application Publication
(10) Pub. No.: US 2015/0247125 A1
(43) Pub. Date: Sep. 3, 2015

(54) MICRORNAS AND CELLULAR REPROGRAMMING

(71) Applicant: LONZA WALKERSVILLE, Walkersville, MD (US)

(72) Inventors: Inbar Friedrich Ben Nun, Rockville, MD (US); Thomas Fellner, Gaithersburg, MD (US)

(73) Assignee: LONZA WALKERSVILLE, Walkersville, MD (US)

(21) Appl. No.: 14/439,245

(22) PCT Filed: Nov. 1, 2013
(86) PCT No.: PCT/US13/68076

§ 371 Date: Apr. 29, 2015

Related U.S. Application Data
(60) Provisional application No. 61/721,990, filed on Nov. 2, 2012.

(51) Int. Cl. C12N 5/074

(52) U.S. Cl. CPC C12N 5/0696; C12N 2501/65; C12N 2506/11; C12N 2500/90; C12N 2501/603; C12N 2501/602; C12N 2501/604; C12N 2501/60; C12N 2501/608

(57) ABSTRACT

Methods and compositions for improved cellular reprogramming to generate induced pluripotent stem cells.

### CROSS REFERENCE TO RELATED APPLICATION

This application claims priority to U.S. Provisional Application Ser. No. 61/721,990, filed Nov. 2, 2012, the content of which is herein incorporated by reference in its entirety.

### FIELD

Methods, compositions, and kits to improve cellular reprogramming using microRNAs are described.

### BACKGROUND

Stem cells are ideal tools to understand disease and develop new treatments; however, they can be difficult to obtain in necessary quantities.

The transformation of differentiated cells to induced pluripotent stem cells (iPSCs) has revolutionized stem cell biology by providing a more tractable source of pluripotent cells for regenerative therapy. The derivation of iPSCs from numerous normal and diseased cell sources has enabled the generation of patient-specific stem cells for eventual use in cell therapy and regenerative medicine.

In 2006, Takahashi and Yamanaka (Takahashi K, Yamanaka S. Induction of pluripotent stem cells from mouse embryonic and adult fibroblast cultures by defined factors. Cell. 2006; 126:663-676) demonstrated that differentiated cells can be converted into induced pluripotent stem cells (iPSCs) by the expression of four transcription factors—Oct4, Klf4, Sox2, and c-Myc—which have been termed Yamanaka factors or OKSM factors. A number of alternatives and refinements to the original four-factor reprogramming method have been devised over the years. These have included ectopic expression of alternative reprogramming factors, such as Nanog and Lin28, manipulation of pathways that act as barriers to reprogramming, such as p53 and p21, transient expression of reprogramming proteins to avoid stable genetic modification, and inclusion of chemical inhibitors that increase the efficiency of the reprogramming process (Plath K, Lowry W E. Nat Rev Genet. 12:253-265. 2011). Although there are several alternatives to some of the OKSM factors, including the use of other transcription factors, signaling factors, and pharmacological molecules. However, at least one pluripotent stem cell transcription factor—usually Oct4—is required for efficient iPSC reprogramming (Huangfu et al. Nat. Biotechnol. 26, 795-797. 2008; Huangfu, D. et al Nat. Biotechnol. 26, 1269-1275. 2008; Judson et al., Nat. Biotechnol. 27, 459-461. 2009; Melton et al., Nature 463, 621-626. 2010; Yoshida et al., Cell Stem Cell 5, 237-241.2009). Reprogramming therefore largely remains dependent on the delivery and exogenous expression of one or more of the original Yamanaka factors.

The current standard strategy for iPSC generation relies upon ectopic expression of Oct4, Sox2, Klf4, and Myc (OSKM). The first generation of this strategy used retroviral and/or lentiviral delivery systems to transfect the host cell with DNA encoding the transcription factors. However, this method results in integration of the viral vector into the host genome, which is strongly disfavored. A second generation strategy therefore sought to transiently transfect the host cell using adenovirus, plasmid DNA, episomal DNA, or mini-circle DNA. However, this strategy also has the potential to have foreign nucleic acids integrated in the host genome. The method also requires screening of the cells for integration of the episomal vector into their genome. Genomic DNA is harvested and PCR performed with primers that are specific for a region on the episomal vector. Cells that are found negative are those that are continued with.

Although powerful, there are several limitations to traditional iPSC generation, including numerous steps and the rather low efficiency of the process (0.2%-1.0%) and the necessity of forced expression of at least one pluripotent stem cell transcription factor, including Oct4, Nanog, Sox2, Klf4, and/or Myc. These limitations hamper the use of iPSC technology in high throughput formats such as generation of human iPSC clones from large patient populations.

A third generation strategy uses non-DNA based techniques to deliver the requisite transcription factors, including the use of recombinant proteins, mRNA, and/or small molecules. One potential non-DNA based technique involves manipulating microRNA (miRNA) to influence the expression of transcription factors. MiRNAs are small, noncoding RNAs that regulate gene expression through sequence specific hybridization to the 3′ untranslated region (UTR) of messenger RNA, thereby silencing the gene by either blocking translation or directing degradation of their target messenger RNAs. MiRNA are involved in regulation of many critical biological processes, including cell proliferation, differentiation, apoptosis, morphogenesis, tumor genesis, and metabolism. Human embryonic stem cells (“hESC”) are known to express a unique set of miRNA, over-expressing oncogenic miRNAs and under-expressing tumour suppressor miRNA relative to differentiated cells.

MiRNA can be manipulated to either increase or decrease expression of a gene targeted by the miRNA. Expression of the target gene can be decreased by ectopically expressing the miRNA in a cell. The ectopically expressed miRNA then hybridize to its target mRNA, thereby down-regulating translation. Alternatively, anti-miRNA oligonucleotides can be ectopically expressed in the cell. Anti-microRNA are short oligonucleotides rationally designed to hybridize to an miRNA, thereby inhibiting hybridization of the miRNA to its target.

Recently, several miRNAs of these hESC miRNA have been shown to enhance iPSC reprogramming when expressed along with combinations of the OSKM factors (Judson et al., Nat. Biotechnol. 27, 459-461. 2009). These miRNAs belong to families of miRNAs that are expressed preferentially in embryonic stem cells and are thought to help maintain the ESC phenotype.

It would be beneficial to develop strategies to integrate miRNA manipulation into protocols for generating iPSCs.

It also would be beneficial to have systems for identifying new miRNA for use in generating iPSCs.

### SUMMARY OF THE INVENTION

The present disclosure provides methods, compositions, and kits useful in cellular reprogramming to generate induced pluripotent stem cells.

A method for generating induced pluripotent stem cells is provided, the method comprising introducing at least one nucleic acid encoding an miRNA and/or at least one nucleic acid encoding an anti-miRNA into a differentiated cell, and treating the differentiated cell under conditions suitable for development of an iPSC. The miRNA may be include but is not limited to miR302a, miR302b, miR302c, miR302d, miR372, miR367(3p), miR367(5p). The anti-miRNA may include but is not limited to inhibitor for Let7c, inhibitor for miR29a.

An iPSC culture is also provided, the iPSC culture being obtained by a method comprising introducing at least one nucleic acid encoding an miRNA and/or at least one nucleic acid encoding an anti-miRNA into a differentiated cell, and treating the differentiated cell under conditions suitable for development of an iPSC.

A kit for reprogramming cells to generate iPSCs is also provided, the kit comprising at least one nucleic acid encoding an miRNA and/or at least one nucleic acid encoding an anti-miRNA, and a suitable delivery system. The delivery system may be but is not limited to transformation and transfection.

A method for identifying miRNA capable of inducing expression of factors involved in inducing development of a pluripotent phenotype is also provided, the method comprising monitoring expression of miRNA in human embryonic stem cells (“hESC”) and isolating miRNA that are over-expressed in the hESC relative to differentiated cells.

A method for identifying miRNA capable of inhibiting expression of factors involved in inducing development of a pluripotent phenotype, the method comprising the method comprising monitoring expression of miRNA in a differentiated cell and isolating miRNA that are over-expressed in the differentiated cell relative to hESC.

Other aspects and embodiments will be apparent in light of the following description, examples, and figures.

### BRIEF DESCRIPTION OF THE FIGURES

FIG. 1 shows a schematic of the theoretical role of micro-RNAs in pluripotency and differentiation.

FIG. 2 depicts a schematic of the proposed mechanism of action of microRNAs in cellular reprogramming.

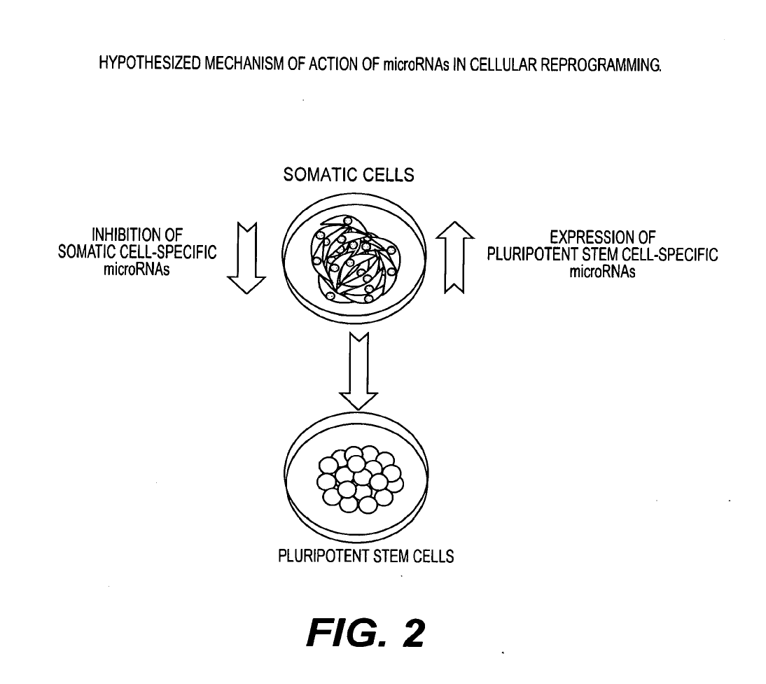

FIG. 3 shows the results from experiments testing the effect of miRNA/anti-miRNA on reprogramming efficiency of C5 & Tg in feeder-free conditions.

FIG. 4 shows the efficiency of reprogramming using miRNAs is significantly enhanced in feeder conditions.

### DETAILED DESCRIPTION

The present disclosure provides compositions, methods and kits useful reprogramming cells to generate induce pluripotent stem cells without the use of DNA elements

A method for generating induced pluripotent stem cells, the method comprising contacting a differentiated cell with a set of reprogramming factors comprising at least one miRNA and/or at least one anti-miRNA and/or at least one nucleic acid encoding an miRNA and/or an anti-miRNA under conditions sufficient for the at least one miRNA and/or at least one anti-miRNA and/or at least one nucleic acid encoding an miRNA and/or an anti-miRNA to enter the cell, and treating the differentiated cell under conditions suitable for development of an iPSC.

In an aspect, the differentiated cell is a cord blood CD34⁺ cell.

In an aspect the miRNA is an miRNA that is over-expressed in a human embryonic stem cell.

In an aspect, the at least one miRNA is an miRNA that hybridizes to or is predicted to hybridize to an mRNA selected from the group consisting of CDKN1A, DOT1L, and SUV39H1.

In an aspect, the at least one miRNA is selected from the group of miRNAs that are highly expressed in human embryonic stem cells. By way of example, the miRNA can include but is not limited to consisting of miR-302 (a, b, c & d), miR-367(3p & 5p), and cmiR372.

In an aspect, the at least one anti-miRNA hybridizes to or is predicted to hybridize to an miRNA selected from the group consisting of Let7 and miR-29. In another aspect, the anti-miRNA hybridizes to an miRNA that hybridizes to or is predicted to hybridize to an mRNA selected from the group consisting of MYC, LIN28, BCL2, DNM3B, DNM3A, BCL2, and CDK6.

In an aspect, the at least one anti-miRNA hybridizes to or is predicted to hybridize to an miRNA that is highly expressed in somatic cells. By way of example, the anti-miRNA can include but is not limited to Anti-Let7a and Anti-miR29a.

In an aspect, the differentiated cell is further contacted with at least one additional reprogramming factor. As used herein, the term “reprogramming factor” refers to any entity that can participate in the transformation of a differentiated cells into an induced pluripotent stem cells. Reprogramming factors include but are not limited to microRNAs, anti-microRNAs, and other factors, such as Oct4, Sox2, Klf4, Myc, Lin28, and SV40 Large T Antigen), and/or nucleic acids encoding the same.

In an aspect, a combination of miRNA and/or anti-miRNA are selected to replace at least one reprogramming factor. In an aspect, a group of miRNA and anti-miRNA is selected to replace SV40 Large T Antigen in a reprogramming protocol. In an aspect, the group of miR-302 (a, b, c & d), miR-367(3p & 5p), miR372, Anti-Let7a and Anti-miR29a replaces SV40 Large T Antigen.

In an aspect, the reprogramming factors can be in the form of an episomal vector, nucleic acid, or protein.

In an aspect, the method comprises contacting the differentiated cell with Oct4, Sox2, Klf4, Myc, Lin28, miR-302 (a, b, c & d), miR-367(3p & 5p), miR372, Anti-Let7a and Anti-miR29a, and optionally SV40 Large T Antigen, or nucleic acids encoding the same.

In another aspect, the differentiated cell is further contacted with entities that aid with uptake of the reprogramming factors, such as but not limited to transformation and transfection, or is manipulated in a manner that aids with uptake of the reprogramming factors, such as by electroporation. A person of ordinary skill in the art would be able to select an appropriate entity or manipulation, depending on the cell type to be used and the type of reprogramming factor to be delivered. In an aspect, the entity or manipulation is suitable for delivery of an episomal vector to a cord blood CD34⁺ cell. As an example, the episomal vector is delivered to a cord blood CD34⁺ cell using P3 4D-NUCLEOFECTOR™ X Solution (Lonza, Basel, CH) and the 4D NUCLEOFECTOR™ system (Lonza, Basel, CH). The LONZA 4D NUCLEOFECTOR™ system uses a technology based on the momentary creation of small pores in cell membranes by applying an electrical pulse. The comprehensive way in which NUCLEOFECTOR™ Programs and cell type-specific solutions are developed enables nucleic acid substrates delivery not only to the cytoplasm, but also through the nuclear membrane and into the nucleus. This allows for high transfection efficiencies up to 99% and makes the transfection success independent from any cell proliferation.

In a further aspect, the method is performed without feeder cells using a zeno-free, cGMP compatible medium.

A culture medium for generating induced pluripotent stem cells is also provided, said culture medium comprising a set of reprogramming factors comprising at least one miRNA and/or at least one anti-miRNA and/or at least one nucleic acid encoding an miRNA and/or an anti-miRNA under conditions sufficient for the at least one miRNA and/or at least one anti-miRNA and/or at least one nucleic acid encoding an miRNA and/or an anti-miRNA to enter the cell, and optionally other factors suitable for development and growth of an iPSC.

In an aspect the miRNA is an miRNA that is over-expressed in a human embryonic stem cell.

In an aspect, the at least one miRNA is an miRNA that hybridizes to or is predicted to hybridize to an mRNA selected from the group consisting of CDKN1A, DOT1L, and SUV39H1.

In an aspect, the at least one miRNA is selected from the group of miRNAs that are highly expressed in human embryonic stem cells. By way of example, the miRNA can include but is not limited to consisting of miR-302 (a, b, c & d), miR-367(3p & 5p), and cmiR372.

In an aspect, the at least one anti-miRNA hybridizes to or is predicted to hybridize to an miRNA selected from the group consisting of Let7 and miR-29. In another aspect, the anti-miRNA hybridizes to an miRNA that hybridizes to or is predicted to hybridize to an mRNA selected from the group consisting of MYC, LIN28, BCL2, DNM3B, DNM3A, BCL2, and CDK6.

In an aspect, the at least one anti-miRNA hybridizes to or is predicted to hybridize to an miRNA that is highly expressed in somatic cells. By way of example, the anti-miRNA can include but is not limited to Anti-Let7a and Anti-miR29a.

In an aspect, the culture medium further comprises at least one additional reprogramming factor. Reprogramming factors include but are not limited to microRNAs, anti-microRNAs, and other factors, such as Oct4, Sox2, Klf4, Myc, Lin28, and SV40 Large T Antigen), and/or nucleic acids encoding the same.

In an aspect, the reprogramming factors are in the form of an episomal vector, nucleic acid, or protein.

In an aspect, the culture medium comprises Oct4, Sox2, Klf4, Myc, Lin28, miR-302 (a, b, c & d), miR-367(3p & 5p), miR372, Anti-Let7a and Anti-miR29a, and optionally SV40 Large T Antigen, or nucleic acids encoding the same.

In an aspect, a combination of miRNA and/or anti-miRNA is selected to replace at least one reprogramming factor. In an aspect, a group of miRNA and anti-miRNA is selected to replace SV40 Large T Antigen in a reprogramming protocol. In an aspect, the group of miR-302 (a, b, c & d), miR-367(3p & 5p), miR372, Anti-Let7a and Anti-miR29a replaces SV40 Large T Antigen.

In another aspect, the culture medium further comprises entities that aid with uptake of the reprogramming factors, such as entities that aid with transfection. A person of ordinary skill in the art would be able to select an appropriate entity, depending on the cell type to be used and the type of reprogramming factor to be delivered. In an aspect, the entity is suitable for delivery of episomal vectors to a cord blood CD34⁺ cell, such as P3 4D-NUCLEOFECTOR™ X Solution (Lonza, Basel, CH).

In a further aspect, the culture medium is suitable for generating iPSCs without feeder cells using for instance a zeno-free, cGMP compatible medium.

A kit for reprogramming cells is also provided, the kit comprising reprogramming factors, and, optionally, a transformation medium. The factors and media supplement may be provided as individual components, as pre-mixes with one or more of the other components of the kits, or as a premixed cell culture medium. The components of the kits may be provided as concentrated component stocks or premixed component stock, as a concentrated cell culture medium, or as a cell culture medium at working concentrations.

In an aspect, the kits may comprise a set of reprogramming factors comprising at least one miRNA and/or at least one anti-miRNA and/or at least one nucleic acid encoding an miRNA and/or an anti-miRNA under conditions sufficient for the at least one miRNA and/or at least one anti-miRNA and/or at least one nucleic acid encoding an miRNA and/or an anti-miRNA to enter the cell.

In an aspect, the at least one miRNA is capable of hybridizing to an mRNA selected from the group consisting of CDKN1A, DOT1L, SUV39H1.

In an aspect, the at least one miRNA is selected from a group that is highly expressed in human embryonic stem cells. By way of example, this may include but is not limited to miR-302 (a, b, c & d), miR-367(3p & 5p), miR372.

In an aspect, the at least one anti-miRNA is capable of hybridizing to an miRNA selected from the group consisting of Let7 and miR-29. In another aspect, the anti-miRNA targets an miRNA capable of hybridizing to an mRNA selected from the group consisting of MYC, LIN28, BCL2, DNM3B, DNM3A, BCL2, and CDK6.

In an aspect, the at least one anti-miRNA is selected from a group that is highly expressed in somatic cells. By way of example, this may include but is not limited to Anti-Let7a and Anti-miR29a.

In an aspect, the kit further comprises with at least one additional reprogramming factor. In a further aspect, the additional reprogramming factor is selected from the group consisting of Oct4, Sox2, Klf4, Myc, Lin28, and SV40 Large T Antigen), and/or nucleic acids encoding the same.

In an aspect, the culture medium further comprises at least one additional reprogramming factor in the form of an episomal vector, nucleic acid, or protein.

In an aspect, the kit comprises Oct4, Sox2, Klf4, Myc, Lin28, miR-302 (a, b, c & d), miR-367(3p & 5p), miR372, Anti-Let7a and Anti-miR29a, and optionally SV40 Large T Antigen, or nucleic acids encoding the same.

In another aspect, the kit further comprises at least one entity that aids with uptake of the reprogramming factors by a cell, such as entities that aid with transformation or transfection.

In a further aspect, the kit comprises a culture medium or stocks useful in making a culture medium. In a further aspect, the culture medium is suitable for generating iPSCs without feeder cells for instance using zeno-free, cGMP compatible medium.

The following examples are illustrative only. Other aspects and embodiments will be readily apparent in light of the present description, examples, and figures.

### EXAMPLES

### Example 1

### Use of miRNA in Combination with Anti-miRNA in the Presence or Absence of Further Reprogramming Factors

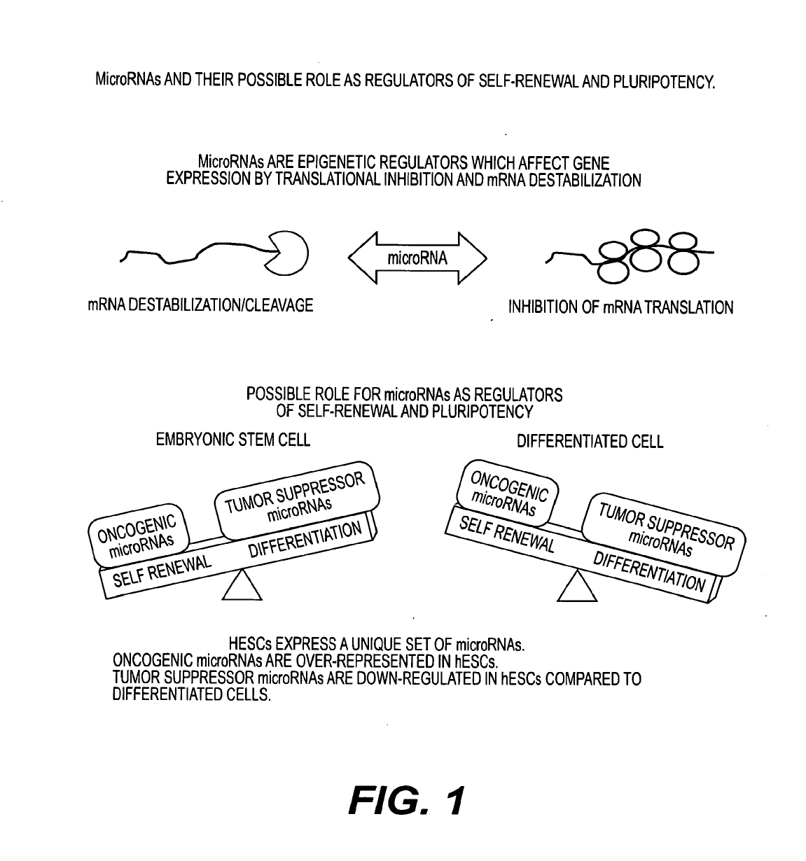

As depicted in FIG. 1, natural miRNA expression is believed to influence the differentiation status of a given cell. hESCs have been shown to express a distinct set of miRNA from differentiated cells. It therefore was hypothesized that a combination of miRNA correlating with hESC and anti-miRNA specific for miRNA correlating with somatic cells could increase the efficiency at which iPSCs could be generated. See FIG. 1.

### Example 2

### Reprogramming of Human Cord Blood CD34+ Cells with Episomal Vectors on Feeders

The following experimental procedure was utilized:

Materials

Serum free medium (SFM) [50% IMDM, 50% Ham's F12, 1:100 Chemical defined synthetic lipid, 1×ITS-X supplement (insulin-transferrin-selenium), 50 μg/ml ascorbic acid, 5 mg/ml BSA, 2 mM glutamine];
Cytokines: 100 ng/ml SCF, 100 ng/ml FL, 20 ng/ml TPO, 10 ng/ml IL-3
MEF medium: [DMEM, 10% FBS]
hESC medium [Knockout DMEM/F12 medium, 20% Knockout serum replacer, 1×NEAA, 55 nM β-Mercaptoethanol, 10 ng/ml bFGF

### MEF-Conditioned Medium

1. Coat T75 flask with gelatin at least one day before seeding cells.
2. Seed 5×10⁶ MEF cells into one T75 flask in 20 ml MEF medium and let attach overnight.
3. Remove medium, wash once with 1×PBS and add 20 ml hESC medium for overnight incubation.
4. On the next day collect conditioned medium and store at 4° C.
5. Collect conditioned medium daily for 7 days before discarding MEFs.
6. Combine collected media, filter sterile and store at −20° C. Add fresh bFGF (f.c. 10 ng/ml) before using.

### Other Reagents

### Gelatine

Methods

Step 1: Revive and Expand Human CD34+ Cells for 4-5 Days

### Day 0

Thaw 1 vial of human cord blood CD34+ cells (˜1,000,000 cells) into 1 well in a 12-well plate and culture in 1 ml serum-free medium (SFM) supplemented with cytokines (100 ng/ml SCF, 100 ng/ml FL, 20 ng/ml TPO, 10 ng/ml IL-3) for 4-5 days to prime the cells.

### Day 2

Collect the cells and re-plate in 2 wells in a 12-well plate. Add fresh 0.5 ml SFM to each well.

Step 2: Reprogramming Human CD34+ Cells

### Day 0

1. Collect cells in a 15 ml conical tube. Count cells. Place 10⁶ cells in a new tube and pellet the cells (90×g, 5′). Remove medium and resuspend cells with premixed Nucleofection™ Solution: 100 μl Primary cell p3 containing 10 μg episomal vectors (8 μg pEB-C5+2 μg pEB-Tg or 8 μg pEB-C5 only). Mix, and transfer to a Nucleofection™ cuvette.
2. Subject cells to 4D NUCLEOFECTOR™
3. After treating with the 4D NUCLEOFECTOR™, using transfer pipet add 500 μl prewarmed SFM and transfer the cells to 1 well in a 12-well plate containing 1.5 ml prewarmed SFM. Place cells in hypoxic (3% O₂ incubator)

### Day 1

Coat 6-well plate with 0.1% gelatin and seed MEF feeders (Millipore Cat #PMEF-CF) following manufacturer's suggested protocol.

### Day 2

Collect nucleofected CD34+ cells and spin down. Resuspend cells in 6 ml MEF medium and seed onto MEF feeders in 1 well of one 6-well plate. Place cells in hypoxic (3% O₂ incubator).

### Day 3

Change into hESC medium. Change fresh hESC medium every other day.

### Day 10

Culture cells in MEF-conditioned medium (MEF-CM) since day 10 till colonies are large enough for picking up.
iPSC colonies should be visible on Day 14 to Day 16.

### Example 3

### Reprogramming of Human Cord Blood CD34+ Cells with Episomal Vectors on Xeno-Free, cGMP Compatible Conditions

The following experimental procedure was utilized.

Materials

Serum free medium (SFM): [50% IMDM, 50% Ham's F12, 1:100 Chemical defined synthetic lipid, lx ITS-X supplement (insulin-transferrin-selenium), 50 ug/ml ascorbic acid, 5 mg/ml BSA, 2 mM glutamine

Cytokines: 100 ng/ml SCF, 100 ng/ml FL, 20 ng/ml TPO, 10 ng/ml IL-3
MEF medium [DMEM, 10% FBS]
Xeno-free, cGMP compatible, medium

### Vitronectin

Methods:

Step 1: Revive and Expand Human CD34+ Cells for 4-5 Days

### Day 0

Thaw 1 vial of human cord blood CD34+ cells (˜1,000,000 cells) into 1 well in a 12-well plate and culture in 1 ml serum-free medium (SFM) supplemented with cytokines (100 ng/ml SCF, 100 ng/ml FL, 20 ng/ml TPO, 10 ng/ml IL-3) for 4-5 days to prime the cells.

### Day 2

Collect the cells and re-plate in 2 wells in a 12-well plate. Add fresh 0.5 ml SFM to each well.

Step 2: Reprogramming Human CD34+ Cells

### Day 0

1. Collect cells in a 15 ml conical tube. Count cells. Place 10⁶ cells in a new tube and pellet the cells (90×g, 5′). Remove medium and resuspend cells with premixed Nucleofection™ Solution: 100 μl Primary cell p3 containing 10 μg episomal vectors (8 μg pEB-C5+2 μg pEB-Tg or 8 μg pEB-C5 only). Mix, and transfer to 10 wells in a Nucleofection™ cuvette.
2. Subject cells to 4D NUCLEOFECTOR™
3. After treating with the 4D NUCLEOFECTOR™, using transfer pipet add 500 μl prewarmed SFM and transfer the cells to 1 well in a 12-well plate containing 1.5 ml prewarmed SFM. Place cells in hypoxic (3% O₂ incubator).

### Day 1

Coat 6-well plate with Vitronectin.

### Day 2

Collect nucleofected CD34+ cells and spin down. Resuspend cells in Zeno-free, cGMP compatible, medium. Seed cells onto one vitronectin coated well in one 6-well plate. Place cells in hypoxic (3% O₂ incubator). Change medium every other day. iPSC colonies should be visible on Day 8 to Day 10.

### Example 4

### Reprogramming of Human Cord Blood CD34+ Cells with Episomal Vectors and microRNAs on Xeno-Free, cGMP Compatible Conditions

The following experimental procedure was utilized.

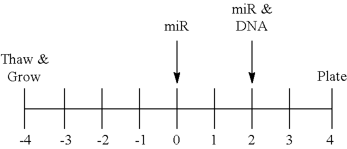

Materials:

Serum free medium (SFM): 50% IMDM, 50% Ham's F12, 1:100 Chemical defined synthetic lipid, 1×ITS-X supplement (insulin-transferrin-selenium), 50 μg/ml ascorbic acid, 5 mg/ml BSA, 2 mM glutamine.
Cytokines: 100 ng/ml SCF, 100 ng/ml FL, 20 ng/ml TPO, 10 ng/ml IL-3.
MEF medium: DMEM, 10% FBS.
Lonza xeno-free, cGMP compatible, medium.

### Vitronectin.

Step 1: Revive and Expand Human CD34+ Cells for 4-5 Days

### Day 0

Thaw 1 vial of human cord blood CD34+ cells (˜1,000,000 cells) into 1 well in a 12-well plate and culture in 1 ml serum-free medium (SFM) supplemented with cytokines (100 ng/ml SCF, 100 ng/ml FL, 20 ng/ml TPO, 10 ng/ml IL-3) for 4-5 days to prime the cells.

### Day 2

Collect the cells and re-plate in 2 wells in a 12-well plate. Add fresh 0.5 ml SFM to each well.

Step 2: Reprogramming Human CD34+ Cells

### Day 0

1. Collect cells in a 15 ml conical tube. Count cells. Place 10⁶ cells in a new tube and pellet the cells (90×g, 5′). Remove medium and resuspend cells with premixed NUCLEOFECTION™ Solution: 200 μl Primary cell p3 containing 50 nmol of each microRNA. Mix, and transfer to 10 wells in a NUCLEOFECTION™ strip (20 μl/well. 10⁵ cells/well).
2. Subject cells to 4D NUCLEOFECTOR™ on program EO-117.
3. After treatment with 4D NUCLEOFECTOR™, add 80 μl prewarmed SFM to each Nucleofection™ well and transfer the cells from 10 wells to 1 well in a 12-well plate containing 1.5 ml prewarmed SFM. Place cells in hypoxic (3% O₂) incubator.

### Day 1

Coat 6-well plate with Vitronectin.

### Day 2

1. Collect cells in a 15 ml conical tube. Count cells. Place 10⁶ cells in a new tube and pellet the cells (90×g, 5′). Remove medium and resuspend cells with premixed Nucleofection™ Solution: 200 μl Primary cell p3 containing 10 μg episomal vectors (8 μg pEB-C5+2 μg pEB-Tg or 8 μg pEB-C5 only) and 50 nmol of each microRNA. Mix, and transfer to 10 wells in a Nucleofection™ strip (20 μl/well).
2. Adjust cell number to have 10⁵ cells/well. Adjust Nucleofection™ Solution, episomal vectors and miRs to cell number.
3. Subject cells to 4D NUCLEOFECTOR™.
4. After treatment with 4D NUCLEOFECTOR™, add 80 μl prewarmed SFM to each Nucleofection™ well and transfer the cells from 10 wells to 1 well in a 12-well plate containing 1.5 ml prewarmed SFM. Place cells in hypoxic (3% O₂) incubator.

### Day 4

Collect nucleofected CD34+ cells and spin down. Resuspend cells in Zeno-free, cGMP compatible, medium. Seed cells onto one vitronectin coated well in one 6-well plate. Place cells in hypoxic (3% O₂) incubator. Change medium every other day. iPSC colonies should be visible on Day 10 to Day 14.

### Example 5

### Reprogramming of Human Cord Blood CD34+ Cells with Episomal Vectors and microRNAs on Feeders

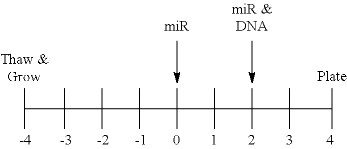

The following experimental procedure was utilized.

### Materials:

Serum free medium (SFM) [50% IMDM, 50% Ham's F12, 1:100 Chemical defined synthetic lipid, 1×ITS-X supplement (insulin-transferrin-selenium), 50 ug/ml ascorbic acid, 5 mg/ml BSA, 2 mM glutamine]
Cytokines [100 ng/ml SCF, 100 ng/ml FL, 20 ng/ml TPO, 10 ng/ml IL-3]
MEF medium [DMEM, 10% PBS]
hESC medium [Knockout DMEM/F12 medium, 20% Knockout serum replacer, 1×NEAA, 55 nM β-Mercaptoethanol, 10 ng/ml bFGF]
MEF-conditioned medium
1. Coat T75 flask with gelatin at least one day before seeding cells.
2. Seed 5×10⁶ MEF cells into one T75 flask in 20 ml MEF medium and let attach overnight.
3. Remove medium, wash once with 1×PBS and add 20 ml hESC medium for overnight incubation.
4. On the next day collect conditioned medium and store at 4° C.
5. Collect conditioned medium daily for 7 days before discarding MEFs.
6. Combine collected media, filter sterile and store at −20° C. Add fresh bFGF (f.c. 10 ng/ml) before using.

### Step 1: Revive and Expand Human CD34+ Cells for 4-5 Days

### Day 0

Thaw 1 vial of human cord blood CD34+ cells (˜1,000,000 cells) into 1 well in a 12-well plate and culture in 1 ml serum-free medium (SFM) supplemented with cytokines (100 ng/ml SCF, 100 ng/ml FL, 20 ng/ml TPO, 10 ng/ml IL-3) for 4-5 days to prime the cells.

### Day 2

Collect the cells and re-plate in 2 wells in a 12-well plate. Add fresh 0.5 ml SFM to each well.

### Step 2: Reprogramming Human CD34+ Cells

### Day 0

1. Collect cells in a 15 ml conical tube. Count cells. Place 10⁶ cells in a new tube and pellet the cells (90×g, 5′). Remove medium and resuspend cells with premixed Nucleofection™ Solution: 200 μl Primary cell p3 containing 50 nmol of each microRNA. Mix, and transfer to 10 wells in a Nucleofection™ strip (20 μl/well. 10⁵ cells/well).
2. Subject cells to 4D NUCLEOFECTOR™ on program EO-117.
3. After treatment with 4D NUCLEOFECTOR™, add 80 μl prewarmed SFM to each Nucleofection™ well and transfer the cells from 10 wells to 1 well in a 12-well plate containing 1.5 ml prewarmed SFM. Place cells in hypoxic (3% O₂ incubator).

### Day 1

Coat 6-well plate with 0.1% gelatin and seed MEF feeders (Millipore Cat#PMEF-CF) following manufacturer's suggested protocol.

### Day 2

1. Collect cells in a 15 ml conical tube. Count cells. Place 10⁶ cells in a new tube and pellet the cells (90×g, 5′). Remove medium and resuspend cells with premixed Nucleofection™ Solution: 200 μl Primary cell p3 containing 10 μg episomal vectors (8 μg pEB-C5+2 μg pEB-Tg or 8 μg pEB-C5 only) and 50 nmol of each microRNA. Mix, and transfer to 10 wells in a Nucleofection™ strip (20 μl/well).
2. Adjust cell number to have 10⁵ cells/well. Adjust Nucleofection™ Solution, episomal vectors and miRs.
3. Subject cells to 4D NUCLEOFECTOR™ on program EO-117.
4. After treatment with 4D NUCLEOFECTOR™, add 80 μl prewarmed SFM to each Nucleofection™ well and transfer the cells from 10 wells to 1 well in a 12-well plate containing 1.5 ml prewarmed SFM. Place cells in hypoxic (3% O₂ incubator).

### Day 4

Collect nucleofected CD34+ cells and spin down. Resuspend cells in 2 ml MEF medium and seed onto MEF feeders in 1 well of one 6-well plate. Place cells in hypoxic (3% O₂ incubator).

### Day 5

Change into hESC medium. Change fresh hESC medium every other day

### Day 15

Culture cells in MEF-conditioned medium (MEF-CM) since day 15 until colonies are large enough for picking up.
iPSC colonies should be visible on Day 11 to Day 14.

### Example 6

### Use of miRNA/Anti-miRNA Combination with Various Combinations of Other Reprogramming Factors

The effect of a combination of the following miRNA or anti-miRNA targeting the same:

| | | Predicted Relevant |
|---|---|---|
| | Validated target | Target |
| miRNA | (By miRTarBase) | (By microRNA.org) |
| miR-302 | CDKN1A | DOT1L, SUV39H1, |
| | | TGFβRII |
| miR372 | CDKN1A | TGFβRII |
| miR-367(3p & 5p), | Not determined | |
| Let7 | MYC, LIN28, BCL2 | |
| miR-29 | DNM3B, DNM3A, | |
| | BCL2, CDK6 | |

iPSCs were generated from CD34+ cells with episomal vectors encoding for the following reprogramming factors:

| | C5 | C5 + Tg | C5 + miRs | C5 + Tg + miRs |
|---|---|---|---|---|
| Oct4 | + | + | + | + |
| Sox2 | + | + | + | + |
| Klf4 | + | + | + | + |
| Myc | + | + | + | + |
| Lin28 | + | + | + | + |
| SV-40 Large T antigen | − | + | − | + |
| miR-302 (a, b, c & d) | − | − | + | + |
| miR-367 (3p & 5p) | − | − | + | + |
| miR372 | − | − | + | + |
| Anti-Let7a | − | − | + | + |
| Anti-miR29a | − | − | + | + |

### Example 4

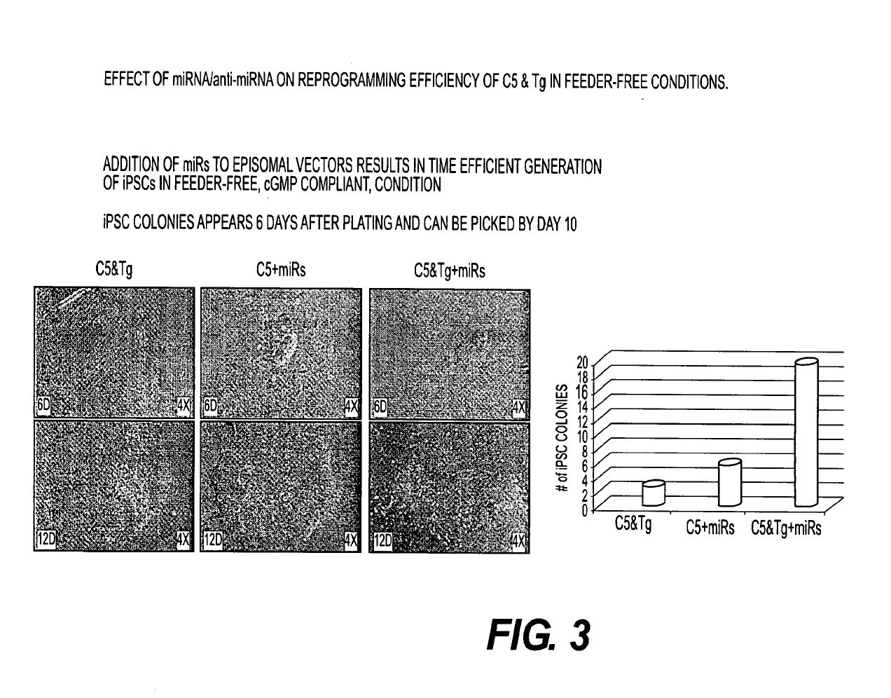

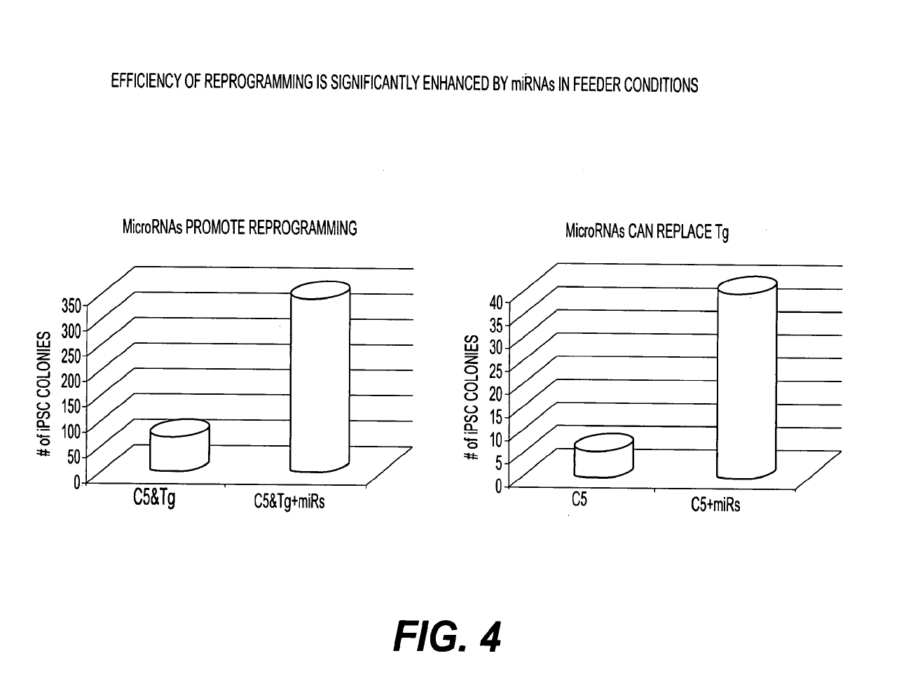

Experiments were conducted to test the efficiency of reprogramming using miRNAs in feeder cell and zeno-free, cGMP compatible medium. See FIGS. 3 and 4. For the feeder cell condition, the number of iPSC colonies was 270 when miRNA was added compared to 45 without miRNA. In zeno-free, cGMP compatible medium, the number of iPSC colonies was 20 when miRNA was added compared to 4 without miRNA. In both instances, reprogramming efficiency is significantly enhanced. Moreover, as shown in the figures, the group of miR-302 (a, b, c & d), miR-367(3p & 5p), miR372, Anti-Let7a and Anti-miR29a is sufficient to replace SV40 Large T Antigen in the protocols.

1. A method for generating induced pluripotent stem cells, the method comprising introducing at least one nucleic acid encoding an miRNA and/or at least one nucleic acid encoding an anti-miRNA into a differentiated cell, and treating the differentiated cell under conditions suitable for development of an iPSC.

2. The method of claim 1, wherein the at least one miRNA is capable of hybridizing to an mRNA selected from the group consisting of CDKN1A, DOT1L, SUV39H1.

3. The method of claim 1, wherein the at least one miRNA is selected from the group consisting of miR-302 (a, b, c & d), miR-367(3p & 5p), miR372.

4. The method of claim 1, wherein the at least one anti-miRNA is capable of hybridizing to an miRNA selected from the group consisting of Let7, miR-29.

5. The method of claim 1, wherein the anti-miRNA targets an miRNA capable of hybridizing to an mRNA selected from the group consisting of MYC, LIN28, BCL2, DNM3B, DNM3A, BCL2, and CDK6.

6. The method of claim 1, wherein the at least one anti-miRNA is selected from the group consisting of Anti-Let7a and Anti-miR29a.

7. The method of claim 1, further comprising with at least one additional reprogramming factor.

8. The method of claim 7, wherein the additional reprogramming factor is selected from the group consisting of Oct4, Sox2, Klf4, Myc, Lin28, and SV40 Large T Antigen, and/or nucleic acids encoding the same.

9. The method of claim 8, wherein the culture medium comprises Oct4, Sox2, Klf4, Myc, Lin28, miR-302 (a, b, c & d), miR-367(3p & 5p), miR372, Anti-Let7a, Anti-miR29a, and optionally SV40 Large T antigen, or nucleic acids encoding the same.

10. An iPSC culture obtained by the method of claim 1.

11. A culture medium for generating induced pluripotent stem cells, the culture medium comprising at least one nucleic acid encoding an miRNA and/or at least one nucleic acid encoding an anti-miRNA into a differentiated cell, and optionally other factors suitable for development and growth of an iPSC.

12. The culture medium of claim 9, wherein the at least one miRNA is capable of hybridizing to an mRNA selected from the group consisting of CDKN1A, DOT1L, SUV39H1.

13. The culture medium of claim 9, wherein the at least one miRNA is selected from the group consisting of miR-302 (a, b, c & d), miR-367(3p & 5p), miR372.

14. The culture medium of claim 9, wherein the at least one anti-miRNA is capable of hybridizing to an miRNA selected from the group consisting of Let7, miR-29.

15. The culture medium of claim 9, wherein the anti-miRNA targets an miRNA capable of hybridizing to an mRNA selected from the group consisting of MYC, LIN28, BCL2, DNM3B, DNM3A, BCL2, and CDK6.

16. The culture medium of claim 9, wherein the at least one anti-miRNA is selected from the group consisting of Anti-Let7a and Anti-miR29a.

17. The culture medium of claim 9, further comprising with at least one additional reprogramming factor.

18. The culture medium of claim 17, wherein the additional reprogramming factor is selected from the group consisting of Oct4, Sox2, Klf4, Myc, Lin28, and SV40 Large T Antigen, and/or nucleic acids encoding the same.

19. The culture medium of claim 18, wherein the culture medium comprises Oct4, Sox2, Klf4, Myc, Lin28, miR-302 (a, b, c & d), miR-367(3p & 5p), miR372, Anti-Let7a, and Anti-miR29a, and optionally SV-40 Large T antigen, or nucleic acids encoding the same.

20. A kit for reprogramming a cell to generate an iPSC, the kit comprising at least one nucleic acid encoding an miRNA and/or at least one nucleic acid encoding an anti-miRNA, and a suitable delivery system.

21. A method for identifying miRNA capable of inducing expression of factors involved in inducing development of a pluripotent phenotype is also provided, the method comprising monitoring expression of miRNA in human embryonic stem cells (“hESC”) and isolating miRNA that are over-expressed in the hESC relative to differentiated cells.

22. A method for identifying miRNA capable of inhibiting expression of factors involved in inducing development of a pluripotent phenotype, the method comprising the method comprising monitoring expression of miRNA in a differentiated cell and isolating miRNA that are over-expressed in the differentiated cell relative to hESC.
